(19) United States
(12) Patent Application Publication
(10) Pub. No.: US 2005/0020658 A1
(43) Pub. Date: Jan. 27, 2005

(54) Selective cyclooxygenase-2 inhibitor patch

(76) Inventors: Katsuyuki Inoo, Itano-gun (JP); Ken-Ichi Hattori, Okawa-gun (JP); Noriko Shimizu, Okawa-gun (JP)

Correspondence Address: PHARMACIA CORPORATION;Global Patent Department, Mail Zone 1006, 575 Maryville Centre Drive, St. Louis, MO, 63141, US

(21) Appl. No.: 10/683,623

(22) Filed: Oct. 10, 2003

Related U.S. Application Data
(60) Provisional application No. 60/428,054, filed on Nov. 21, 2002.

(51) Int. Cl. A61K 31/415; A61K 31/18

(52) U.S. Cl. 514/406; 514/602

(57) ABSTRACT

A pharmaceutical composition for application to an area of skin of a subject for local and/or systemic treatment of a COX-2 mediated disorder comprises a backing sheet that is flexibly conformable to the area of skin, the backing sheet having opposing surfaces that are respectively distal and proximal to the skin when applied; and a coating on the proximal surface of the backing sheet that comprises (a) an adhesive, (b) an active agent comprising a selective COX-2 inhibitory sulfonamide drug of low water solubility, and (c) a solvent system for the active agent, wherein the active agent is in a therapeutically effective total amount and the solvent system is selected with regard to composition and amount thereof to be effective to maintain the active agent substantially completely in solubilized form. A method of local treatment of a site of pain and/or inflammation in a subject comprises applying the composition to a skin surface of the subject, preferably at a locus overlying or adjacent to the site of pain and/or inflammation, and leaving the composition in place for a time period effective to permit delivery of a locally therapeutic amount of the active agent. A method of systemic treatment of a subject having a COX-2 mediated disorder comprises applying the composition to a skin surface of the subject, and leaving the composition in place for a time period effective to permit transdermal delivery of a therapeutic amount of the active agent.

This application claims priority of U.S. provisional application Ser. No. 60/428,054 filed on Nov. 21, 2002.

### FIELD OF THE INVENTION

The present invention relates to pharmaceutical compositions containing a selective cyclooxygenase-2 (COX-2) inhibitory drug, in particular to such compositions in a form of a patch suitable for administration to skin to provide a local or systemic therapeutic effect. A “patch” herein includes tapes, poultices, pads, plasters, cataplasms, dressings and the like that are capable of adhesion to the skin. The invention also relates to processes for preparing such compositions and to methods of treatment comprising administration of such compositions to skin of a subject in need thereof.

### BACKGROUND OF THE INVENTION

Inhibition of cyclooxygenase (COX) enzymes is believed to be at least the primary mechanism by which nonsteroidal anti-inflammatory drugs (NSAIDs) exert their characteristic anti-inflammatory, antipyretic and analgesic effects, through inhibition of prostaglandin synthesis. Conventional NSAIDs such as ketorolac, diclofenac, naproxen and salts thereof inhibit both the constitutively expressed COX-1 and the inflammation-associated or inducible COX-2 isoforms of cyclooxygenase at therapeutic doses. Inhibition of COX-1, which produces prostaglandins that are necessary for normal cell function, appears to account for certain adverse side effects that have been associated with use of conventional NSAIDs. By contrast, selective inhibition of COX-2 without substantial inhibition of COX-1 leads to anti-inflammatory, antipyretic, analgesic and other useful therapeutic effects while minimizing or eliminating such adverse side effects. Selective COX-2 inhibitory drugs have therefore represented a major advance in the art.

Numerous compounds have been reported having therapeutically and/or prophylactically useful selective COX-2 inhibitory effect, and have been disclosed as having utility in treatment or prevention of specific COX-2 mediated disorders or of such disorders in general. Among such compounds are a large number of substituted pyrazolyl benzenesulfonamides as reported in U.S. Pat. No. 5,466,823 to Talley et al., including for example the compound 4-[5-(4-methylphenyl)-3-(trifluoromethyl)-1H-pyrazol-1-yl]benzenesulfonamide, also referred to herein as celecoxib (I), and the compound 4-[5-(3-fluoro-4-methoxyphenyl)-3-difluoromethyl)-1H-pyrazol-1-yl]benzenesulfonamide, also referred to herein as deracoxib (II).

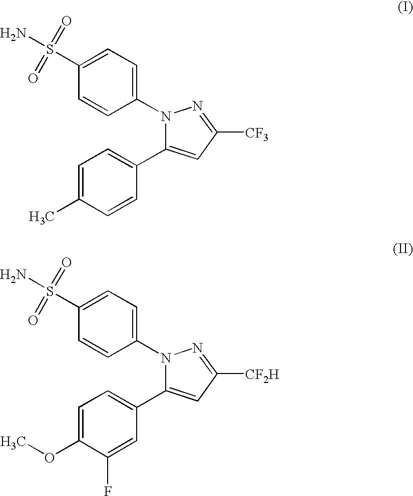

Other compounds reported to have therapeutically and/or prophylactically useful selective COX-2 inhibitory effect are substituted isoxazolyl benzenesulfonamides as reported in U.S. Pat. No. 5,633,272 to Talley et al., including for example the compound 4-[5-methyl-3-phenylisoxazol-4-yl]benzenesulfonamide, also referred to herein as valdecoxib (III).

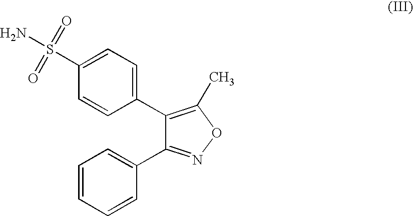

Selective COX-2 inhibitory drugs have been formulated in a variety of ways, principally for oral delivery. However, topical administration of such drugs has been suggested in general terms, for example in some of the above-cited patents.

Above-cited U.S. Pat. No. 5,466,823 and U.S. Pat. No. 5,633,272 disclose that their subject compounds, which include celecoxib and valdecoxib, can be delivered topically.

U.S. Pat. No. 5,932,598 to Talley et al. and U.S. Pat. No. 6,034,256 to Carter et al. disclose that their subject compounds, which are selective COX-2 inhibitors or prodrugs thereof, can be administered by a transdermal device, for example using a patch either of the reservoir and porous membrane type or of a solid matrix variety. In either case, the active agent is said to be delivered continuously from the reservoir or microcapsules through a membrane into an adhesive that is permeable to the active agent, the adhesive being in contact with the skin or mucosa of the recipient.

U.S. Pat. No. 5,208,035 to Ikeda et al. discloses a plaster comprising a backing material and a paste spread thereon. The paste comprises the NSAID diclofenac sodium 1-menthol, propylene glycol and a water-soluble polymer.

U.S. Pat. No. 5,591,767 to Baker et al. discloses a transdermal patch having a depot of the NSAID ketorolac between an occlusive backing layer and a porous membrane. The depot contains, in addition to the ketorolac, a plasticizing-type enhancer selected from isopropyl myristate, caprylic triglyceride, capric triglyceride and glyceryl oleate, and a solvent-type enhancer selected from ethanol, propanol and propylene glycol. An adhesive layer is in contact with the skin-facing side of the porous membrane.

U.S. Pat. No. 5,607,690 to Akazawa discloses an anti-inflammatory and analgesic plaster preparation containing the NSAID diclofenac in the form of its hydroxyethylpyrrolidine salt, which is reported to exhibit enhanced skin permeation by comparison with an otherwise similar preparation containing diclofenac sodium. The low skin permeability of diclofenac sodium is stated therein to result from the low solubility in water of this salt.

U.S. Pat. No. 5,665,378 to Davis & Primo-Davis discloses a transdermal patch formulation comprising an NSAID, the diuretic drug pamabrom, capsaicin and a skin permeation enhancer selected from menthol, eucalyptol, glyceryl monostearate and d-limonene. The formulation is said to be useful for treating menstrual pain.

U.S. Pat. No. 5,916,587 to Jeong et al. discloses a transdermal patch having an adhesive polymer matrix containing the NSAID piroxicam, an absorption assistant (typically a solvent) and a penetration enhancer.

Japanese Patent Publication No. 06-219940 discloses a transdermal patch having a reservoir comprising the NSAID diclofenac sodium in an oil-in-water emulsion.

International Patent Publication No. WO 94/23713 discloses a topical and/or transdermal delivery composition comprising an NSAID, illustratively flurbiprofen, a lipophlic excipient selected from fatty acid alkyl esters and monoglycerides, and a hydrophilic excipient selected from polyethylene glycol, polyethylene glycol esters, isosorbide ethers and diethylene glycol ethers. A pressure sensitive adhesive can be included in the formulation for application to a flexible backing, to form an adhesive-coated sheet material useful as a tape, patch or dressing.

International Patent Publication No. WO 97/29735 discloses a transdermal drug delivery system comprising a dermal penetration enhancer that is an ester sunscreen, preferably a long-chain alkyl ester of p-aminobenzoic acid, dimethyl p-aminobenzoic acid, cinnamic acid, methoxycinnamic acid or salicylic acid, for example octyl dimethyl p-aminobenzoate or octyl salicylate.

Japanese Patent Publication No. 10-114646 discloses a patch comprising an NSAID, illustratively indomethacin, and berberine as an agent to reduce skin irritation.

Japanese Patent Publication No. 10-218793 discloses an adhesive tape comprising a styrene-isoprene-styrene block copolymer, the NSAID felbinac, 1-menthol and oleyl alcohol.

Japanese Patent Publication No. 10-298065 discloses an adhesive tape said to be “warm-feeling”, prepared by laminating a polymer film with a fabric to form a support layer and then laminating with a hydrophilic layer that can contain a blood circulation promoter and an NSAID.

Japanese Patent Publication No. 10-298069 discloses a patch comprising an elastic support having thereon a pressure-sensitive adhesive layer that contains polyether-ester-amide adhesives and an NSAID, illustratively ketoprofen.

Japanese Patent Publication No. 11-199515 discloses a patch comprising an NSAID selected from flurbiprofen, felbinac, bufexamac and suprofen, one or more water-soluble polymers and two or more multivalent metal compounds.

Japanese Patent Publication No. 11-199516 discloses a patch comprising the NSAID flurbiprofen, red pepper extract and a mixture of polymers.

Japanese Patent Publication No. 11-199518 discloses a patch comprising the NSAID flurbiprofen, red pepper extract and β-cyclodextrin.

Japanese Patent Publication No. 11-199519 discloses a patch comprising the NSAID flurbiprofen, red pepper extract and gelatin.

International Patent Publication No. WO 99/62557 discloses a composition for transdermal administration of an NSAID comprising an absorption promoter that consists essentially of a diethylene glycol ether and a sorbitan ester, and an adhesive matrix.

International Patent Publication No. WO 00/41538 discloses a composition for transdermal administration of a drug comprising a blend of two or more acrylic-based polymers having differing functionalities.

International Patent Publication No. WO 00/51575 discloses a transdermal device containing a composition of an NSAID with a skin permeation enhancer selected from fatty alcohols, e.g., oleyl alcohol, and fatty acid esters, e.g., glyceryl monooleate and isopropyl myristate.

Japanese Patent Publication No. 2000/256214 discloses a patch comprising an NSAID and a thermal sense stimulant selected from red pepper extracts, capsaicin and nonanoic acid vanillylamide, formulated in an adhesive base on a silicone-treated polyester film with a polyethylene fabric layered on top.

Korean Patent Publication No. 2000/24702 discloses a poultice comprising the NSAID loxoprofen together with adhesive polymers, auxiliary agents and an absorption accelerator.

European Patent Application No. 1 148 106 discloses a pressure sensitive adhesive tape preparation comprising a drug, e.g., an NSAID, a polyhydric alcohol and a sodium, magnesium, zinc or aluminum salt of a fatty acid.

European Patent Application No. 1 170 020 discloses a composition comprising an NSAID, illustratively diclofenac sodium, and a local anesthetic, illustratively lidocaine, for topical treatment of inflammatory pain, e.g., lumbago. The active agents are reportedly incorporated into an adhesive gel base containing a water-soluble polymer, a cross-linking agent, water and a water holding agent; the gel base is then applied to a nonwoven fabric which is pressed and covered with a polypropylene liner for cutting into patches.

U.S. Pat. No. 6,262,121 to Kawaji & Yamaji discloses an oily patch comprising the NSAID diclofenac sodium, isostearic acid, a fatty acid that is liquid at ambient temperature and an adhesive base.

International Patent Publication No. WO 01/91743 discloses a patch containing, by weight, 0.1-20% of the NSAID 4-biphenylacetic acid (felbinac) together with 5-50% of a styrene/isoprene/styrene block copolymer, 0.05-20% N-methyl-2-pyrrolidone and 0.1-20% polyethylene glycol.

United Kingdom Patent Publication No. 2 362 825 discloses a transdermal patch comprising an NSAID, an alkylpyrrolidone, polyethylene glycol and a hydrophilic nonionic surfactant in an aqueous base that comprises a water-soluble polymer, a water-soluble vinyl polymer and a water-insoluble multivalent metallic salt.

Japanese Patent Publication No. 2002/193793 discloses patch formulations comprising an NSAID such as flurbiprofen. The formulation is prepared by dissolving or dispersing a glycol in a glycerol-containing gel and dispersing the NSAID into the same gel. The gel is then spread on an elastic nonwoven fabric and covered with a polypropylene film to provide a patch.

International Patent Publication No. WO 02/58620 discloses pharmaceutical compositions containing a COX-2 inhibitor, for example a selective COX-2 inhibitor, and a muscle relaxant, illustratively pridinol mesylate. A wide variety of dosage forms is contemplated therein, including a poultice (emplasto) and a patch (parche).

As the foregoing indicates, administration of an adhesive coated sheet comprising an NSAID, in some cases a selective COX-2 inhibitory drug, to the skin with the objective of achieving local or systemic therapeutic effect has been widely contemplated in the art. However, there remains a need in the art for a patch formulation of a selective COX-2 inhibitory drug that can be shown to exhibit a sufficient rate of skin permeation of the drug to achieve such effect.

Where a systemic effect is desired, the composition must be capable of delivering daily an amount of the drug by skin permeation at least equal to the minimum therapeutically effective daily dosage amount when the drug is given orally or parenterally. Furthermore, it is neither practical nor convenient to apply a patch to a very large area of skin to achieve this result; typically a maximum area for application to an adult human subject is about 400 cm², but preferably a much smaller area of skin is treated.

For illustration, in the case of celecoxib, a typical minimum daily dosage amount by oral administration for an adult human is about 200 mg. A minimum permeation rate of 500 μg/cm².day over an area of 400 cm² is therefore needed to provide by transdermal, i.e., systemic, delivery the minimum daily dosage amount of celecoxib. It is generally desirable to treat a much smaller area than 400 cm², thus the minimum permeation rate desired for transdermal delivery is even higher than 500 μg/cm².day. Even where only local, i.e., topical, delivery is desired, a high permeation rate is still important, because the area of skin available for topical application, for example by poultice or tape, is generally no greater than about 140 cm², often less.

Whether a systemic or local therapeutic effect is desired, it has therefore remained a difficult challenge to formulate a selective COX-2 inhibitory drug in a form of a patch providing sufficient permeation to provide therapeutic effectiveness, especially when applied to an area of skin no greater than about 400 cm².

### SUMMARY OF THE INVENTION

There is now provided a pharmaceutical composition for application to an area of skin of a subject for local and/or systemic treatment of a COX-2 mediated disorder. The composition comprises a backing sheet that is flexibly conformable to the area of skin, the backing sheet having opposing surfaces that are respectively distal and proximal to the skin when applied; and a coating on the proximal surface of the backing sheet. The coating comprises (a) an adhesive, (b) an active agent comprising a selective COX-2 inhibitory sulfonamide drug of low water solubility, and (c) a solvent system for the active agent, wherein the active agent is in a therapeutically effective total amount and the solvent system is selected with regard to composition and amount thereofto be effective to maintain the active agent substantially completely in solubilized form.

The selective COX-2 inhibitory sulfonamide drug is a compound having the structural formula (IV): 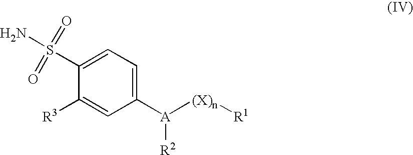
wherein:
A is a substituent selected from partially unsaturated or unsaturated heterocyclic and partially unsaturated or unsaturated carbocyclic rings;
X is O, S or CH₂;
n is 0 or 1;
R¹ is at least one substituent selected from heterocyclyl, cycloalkyl, cycloalkenyl and aryl groups, and is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio groups;
R² is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl groups, R² being optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio groups; and
R³ is selected from hydrido and halo radicals.

In a first preferred embodiment, defined herein as a “tape”, the coating comprises a layer having the active agent dispersed in a lipophilic matrix that comprises the adhesive and the solvent system.

In a second preferred embodiment, defined herein as a “poultice”, the coating comprises a reservoir layer, adjacent to the backing sheet, wherein the active agent is dispersed in a hydrophilic matrix. This layer can also contain the adhesive, but alternatively, a separate adhesive layer overlies the reservoir layer and is proximal to the skin when the poultice is applied thereto. Optionally in such a coating, a membrane that permits passage of the active agent is present between the reservoir layer and the adhesive layer.

In preferred compositions, the coating further comprises one or more skin permeation enhancers.

Preferably a peelable release liner is also provided. This liner, prior to use, is adjacent to the layer that contains the adhesive, and is removed prior to application of the composition to the skin.

There is further provided a method of local treatment of a site of pain and/or inflammation in a subject, the method comprising applying a pharmaceutical composition as provided herein to a skin surface of the subject, preferably at a locus overlying or adjacent to the site of pain and/or inflammation, and leaving the composition in place for a time period effective to permit delivery of a locally therapeutic amount of the active agent.

There is still further provided a method of systemic treatment of a subject having a COX-2 mediated disorder, the method comprising applying a pharmaceutical composition as provided herein to a skin surface of the subject, and leaving the composition in place for a time period effective to permit transdermal delivery of a therapeutic amount of the active agent.

### BRIEF DESCRIPTION OF THE DRAWINGS

FIG. 1 is a schematic drawing in section, not to scale, of a composition of a first embodiment of the invention.

FIG. 2 is a schematic drawing in section, not to scale, of a composition of a second embodiment of the invention.

FIG. 3 is a graph showing skin permeation of celecoxib from 1% solutions thereof in N-methyl-2-pyrrolidone, with and without addition of 1% oleic acid, in a study described in Example 4.

FIG. 4 is a graph showing skin permeation of valdecoxib from 1% solutions thereof in N-methyl-2-pyrrolidone, with and without addition of 1% oleic acid, in a study described in Example 4.

FIG. 5 is a graph showing skin permeation of valdecoxib from various tape formulations and a comparative gel formulation, in a study described in Example 16.

FIG. 6 is a graph showing skin permeation of valdecoxib from various tape formulations and a comparative gel formulation, in a study described in Example 18.

FIG. 7 is a graph showing skin permeation of valdecoxib from poultice formulations with and without polyethylene glycol (PEG) 400, in a study described in Example 23.

FIG. 8 is a graph showing skin permeation of valdecoxib from various poultice formulations, in a study described in Example 25.

FIG. 9 is a graph showing effect of various poultice formulations on swelling in a modified carrageenan-induced paw edema assay described in Example 27.

FIG. 10 is a graph showing effect of various tape formulations on swelling in a modified carrageenan-induced paw edema assay described in Example 28.

### DETAILED DESCRIPTION OF THE INVENTION

The selective COX-2 inhibitory sulfonamide drug is of low water solubility. Preferably the solubility of the drug in water at 25° C. is less than about 10 mg/ml, more preferably less than about 1 mg/ml.

The drug is a compound of formula (IV) as defined above. Herein, unless otherwise indicated, alkyl, alkenyl, alkynyl, alkoxy and acyl groups or subgroups have 1 to about 8, preferably 1 to about 6, carbon atoms, and aryl and heterocyclyl groups are preferably 5- to 6-membered.

Preferably in the compound of formula (IV), A is a pyrazole, furanone, isoxazole, pyridine, cyclopentenone or pyridazinone ring, more preferably a pyrazole or isoxazole ring. In a particularly preferred embodiment the selective COX-2 inhibitory drug is celecoxib (I), deracoxib (II) or valdecoxib (III). Most preferably, the selective COX-2 inhibitory drug is valdecoxib.

The active agent used in compositions of the invention can be prepared by any known process, for example in the case of valdecoxib in the manner set forth in above-cited U.S. Pat. No. 5,633,272, and in the case of celecoxib and deracoxib in the manner set forth in above-cited U.S. Pat. No. 5,466,823.

The active agent is present in an amount and at a concentration sufficient to provide therapeutic efficacy when the composition is applied to the skin and remains in contact therewith for a period of up to about 7 days, preferably up to about 1 day. What constitutes a therapeutically effective amount or concentration depends upon the particular active agent used, the permeability of the skin, the nature of the disorder to be treated, whether local or systemic delivery is required, and other factors.

Typically in the case of valdecoxib, a concentration in the coating of about 0.05% to about 50%, more typically about 0.1% to about 25%, for example about 0.2% to about 10%, by weight is suitable. The amount of valdecoxib per unit area of the composition is typically about 5 to about 5000 μg/cm², more typically about 10 to about 2500 μg/cm², for example about 20 to about 1000 μg/cm². Illustratively, a 10 cm×10 cm (100 cm²) patch containing 200 μg active agent per cm² is equivalent to a 20 mg dose of the active agent, although only a fraction of the applied dose may be transported into and/or through the skin. For example, this illustrative patch may deliver the active agent at a permeation rate of 20 μg/cm².day for 1 day, equivalent to a total delivery of 2 mg of the active agent, or an efficiency of delivery of 2/20, i.e., 10%. Greater and lesser efficiencies of delivery are also within the scope contemplated herein.

A pharmaceutical composition of the invention is described herein as a “patch”, a generic term that will be understood to embrace tapes, poultices, pads, plasters, cataplasms and dressings that are adhesive to skin. The components of the patch are described herein with reference to a skin surface to which the composition is to be applied. As applied to a layer or surface herein, the term “proximal” means toward the skin surface and the term “distal” means away from the skin surface, when the composition is correctly applied.

The most distal layer of the composition is a backing sheet that is flexibly conformable to the skin surface. Any suitable material can be used for the backing sheet, but typically a polymer film, illustratively comprising one or more of polyethylene, polypropylene, polyvinyl chloride, ethylene vinyl acetate copolymer (EVA), polyurethane and polyester, or a woven or nonwoven fabric, e.g., of polyester or rayon, optionally having a polymer film laminated thereon, is used. A presently preferred backing material comprises a nonwoven vinylon fabric laminated with a polyester film as disclosed in U.S. Pat. No. 6,177,098 to Kawaji & Yamaji, incorporated herein by reference. The backing sheet can be airtight and/or waterproof, providing a substantially occlusive dressing. Alternatively, a backing sheet can be used having pores or other means for circulation of air to the treated skin area.

A coating is present on the proximal surface of the backing sheet. As indicated above, the coating comprises (a) an adhesive, (b) an active agent as defined above in a therapeutically effective total amount, and (c) a solvent system selected with regard to composition and amount thereof to be effective to maintain the active agent substantially completely in solubilized form.

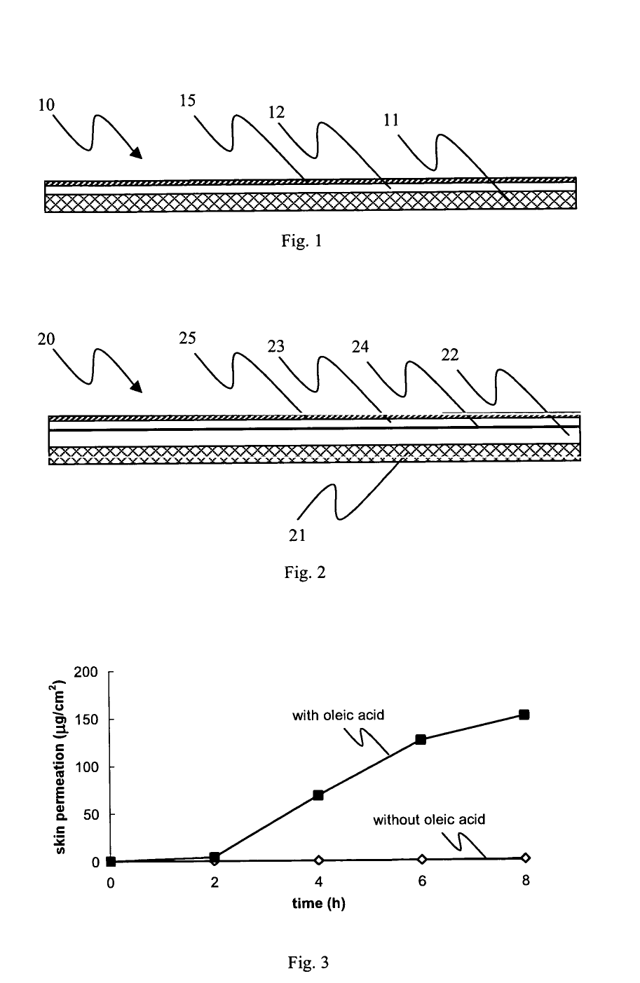

In a first embodiment, the active agent is solubilized in a lipophilic or hydrophilic matrix that comprises the solvent system and the adhesive. As shown in FIG. 1, a composition 10 of this first embodiment comprises a distal backing sheet 11 having on its proximal surface a coating layer 12 wherein the active ingredient is dispersed in solubilized form in the matrix. Where the matrix is lipophilic, the coating is generally relatively thin, e.g., about 50 to about 200 g/m², and the patch is described herein as a “tape”. Where the matrix is hydrophilic, typically an aqueous gel, the coating is generally relatively thick, e.g., about 500 to about 1500 g/m², and the patch is described herein as a “poultice”. On the proximal side of the coating layer 12 is an optional peelable release liner 15 that can be removed to expose the coating layer 12 prior to application to a skin surface.

In a second embodiment, the active agent is solubilized in a solid or semi-solid matrix, for example an aqueous gel, in a reservoir layer adjacent to the backing sheet, and the adhesive is present in a distinct layer proximal to the reservoir layer, optionally with a membrane that permits passage of the active agent between these layers. As shown in FIG. 2, a composition 20 of this second embodiment comprises a distal backing sheet 21 having on its proximal surface a reservoir layer 22 wherein the active ingredient is dispersed in a solid or semi-solid matrix. On the proximal side of the reservoir layer 22 is an adhesive layer 23, optionally separated from the reservoir layer 22 by a membrane 24. On the proximal side of the adhesive layer 23 is an optional peelable release liner 25 that can be removed to expose the adhesive layer 23 prior to application to a skin surface.

Preferably in either of the above embodiments, a release liner is provided. This liner can be made of any suitable material that does not adhere to the adhesive-containing layer or laminated with such a material, so that it is readily peelable without: detaching a significant amount of that layer from the composition. Typical release liners are polyester, polyethylene, polypropylene, PET (polyethylene terephthalate) or polyurethane films laminated with a silicone or fluoropolymer easy-release coating.

The release liner provides some protection for the coating during transport and storage of the composition, but typically the composition is additionally protected by individual packaging, for example a polyethylene wrap. The composition is preferably maintained in sterile condition until the packaging is opened.

The key ingredients of the matrix wherein the active agent is dispersed are the adhesive and the solvent system. Selection of a suitable adhesive, a suitable solvent system and other optional ingredients can be made by one of skill in the art based on the disclosure provided herein, in order to optimize skin permeation of the active agent. In the illustrative lists of ingredients below, certain compounds are listed in more than one class, and it will be recognized that such compounds can serve multiple functions in a composition of the invention, e.g., as adhesive and thickening agent, or as solvent, humectant and skin permeation enhancer.

Preferably the composition exhibits a skin permeation rate of not less than about 1, more preferably not less than about 3 and most preferably not less than about 10 pg/cm².day.

When a skin permeation rate or range of such rates is indicated herein, it will be understood to mean a rate as determined by a standard test, illustratively a standard test using rat skin or human cadaver skin.

As an example of such a test, a Franz diffusion cell can be used having a skin membrane of suitable area, e.g., a disk of diameter 15 mm, and a suitable receptor fluid, for example an N-methylpyrrolidone (NMP) solution. The receptor compartment of the Franz diffusion cell is filled with the receptor fluid and the diffusion cell is maintained at a suitable temperature, preferably a temperature approximating living human skin temperature. A receptor fluid temperature of 32° C. has been found suitable. The membrane is oriented so that its internal surface, i.e., the surface opposite the epidermal surface, is placed in contact with the receptor fluid. Air bubbles are removed from the receptor fluid, which is then allowed to equilibrate with the membrane for a suitable period, typically about 30 minutes. The epidermal surface is dried and a test sample, for example a 10 mm disk, of a composition, with any release layer having been removed, is placed with its adhesive coating in contact with the epidermal surface, and left in place for a desired period, for example 24 hours. It is important to ensure good integrity of contact between the sample and the epidermis. At intervals during this period, and/or at the end of this period, concentration of the active agent is determined in the receptor fluid by a suitable analytical method, e.g., high performance liquid chromatography (HPLC). This concentration is a measure of the amount of the active agent that has permeated the skin membrane during the period of the test, and can be used to calculate a skin permeation rate of active agent in units such as μg/cm².day or μg/cm².hour.

It will be understood that skin membranes exhibit significant variation in permeability, depending on source. Absolute permeation rates through such membranes are therefore less meaningful than permeation rates normalized for permeability of the test membrane used, based on data obtained with a reference composition. A suitable reference composition is a solution of the active agent in 70% aqueous ethanol.

The adhesive generally comprises one or more macromolecular substances. Examples include gelatin, agar, alginic acid, mannan, carboxymethylcellulose, methylcellulose, polyvinyl alcohol, natural rubber, polyisoprene, polybutadiene, polyisobutylene (PIB), styrene-butadiene rubber, styrene-isoprene-styrene (SIS) block copolymers, polyacrylic esters, polymethacrylic esters, acrylic ester-methacrylic ester copolymers, acrylic acid-acrylic ester-vinyl acetate copolymers and petroleum resins. Silicone-based adhesives are another option.

When a natural rubber is used as the base for an adhesive, an illustrative adhesive composition comprises about 30% to about 70% by weight of natural rubber, about 30% to about 60% by weight of a tackifier resin, not more than about 20% by weight of a plasticizer or softening agent and about 0.01% to about 2% of an antioxidant. When the adhesive is based on an SIS block copolymer, an illustrative adhesive composition comprises about 10% to about 30% by weight of the copolymer, about 20% to about 60% by weight of a tackifier resin, about 5% to about 20% by weight of a liquid rubber, about 10% to about 50% by weight of a softening agent and about 0.1% to about 5% by weight of an antioxidant.

Suitable tackifier resins illustratively include alicyclic saturated hydrocarbon petroleum resins, rosin, rosin glycerol ester, hydrogenated rosin, hydrogenated rosin glycerol ester, hydrogenated rosin pentaerythritol ester, cumaroneindene resins, polyterpenes, terpene-phenolic resins, cycloaliphatic hydrocarbon resins, alkyl aromatic hydrocarbon resins, hydrocarbon resins, aromatic hydrocarbon resins and phenolic resins. Suitable liquid rubbers illustratively include polybutene and polyisoprene. Suitable antioxidants illustratively include dibutylhydroxytoluene (BHT). Suitable plasticizers or softening agents illustratively include liquid paraffm and petrolatum (petroleum jelly).

Optionally, a metal sequestering agent can be incorporated into the adhesive composition. Suitable sequestering agents include, among others, ethylene diamine tetraacetic acid (EDTA), potassium polyphosphate, sodium polyphosphate, potassium metaphosphate, sodium metaphosphate, dimethylglyoxime, 8-hydroxyquinoline, nitrilotriacetic acid, dihydroxyethylglycine, gluconic acid, citric acid and tartaric acid. These are illustratively used in an amount of about 0.01% to about 2% by weight.

Selection of adhesive should be made to ensure good “tack”, i.e., adhesion on contact with the skin and maintenance of such contact for the duration of the period for which the patch is to remain in place on the skin. Without good tack, delivery of the active agent into or through the skin can be seriously reduced.

For a tape of the invention, presently preferred adhesives are synthetic rubber systems, for example having a base of SIS copolymer together with a tackifier and softening agent as described above, and polyacrylate systems, particularly hydrocarbon acrylate copolymers. Especially preferred is an SIS based synthetic rubber system. For a poultice of the invention, presently preferred adhesives include polyacrylate, sodium polyacrylate and polyvinyl alcohol.

The solvent system is preferably selected to exhibit good solubility of the active agent therein. Suitable solvents include polyhydric alcohols, for example polyethylene glycols (PEGs), propylene glycol, 1,3-butanediol and dipropylene glycol. PEGs having a molecular weight of about 200 to about 1000, more particularly about 300 to about 600, for example PEG 400, are especially suitable. Other suitable solvents include fatty acid esters, for example isopropyl myristate, diethyl sebacate and diisopropyl adipate (DIA). Yet others include NMP and N-ethyl-N-(2-methylphenyl)-2-butenamide (crotamiton). A presently preferred solvent is NMP.

Optionally, one or more skin permeation enhancers, other than compounds listed above, can be included in the composition.

In one embodiment, a skin permeation enhancer selected from terpenes, terpenoids, fatty alcohols and derivatives thereof is present in the composition. Examples include oleyl alcohol, thymol, menthol, carvone, carveol, citral, dihydrocarveol, dihydrocarvone, neomenthol, isopulegol, 4-terpinenol, menthone, pulegol, camphor, geraniol, α-terpineol, linalool, carvacrol, trans-anethole, isomers thereof and racemic mixtures thereof

Fatty acids such as oleic acid and their alkyl and glyceryl esters such as isopropyl laurate, isopropyl myristate, methyl oleate, glyceryl monolaurate, glyceryl monooleate, glyceryl monostearate, glyceryl dilaurate, glyceryl dioleate, etc. also can be used as skin permeation enhancers. Fatty acid esters of glycolic acid and its salts, for example as disclosed in International Patent Publication No. WO 98/18416, incorporated herein by reference, are also useful skin permeation enhancers. Examples of such esters include lauroyl glycolate, caproyl glycolate, cocoyl glycolate, isostearoyl glycolate, sodium lauroyl glycolate, tromethamine lauroyl glycolate, etc. Also useful as skin permeation enhancers are lactate esters of fatty alcohols, for example lauryl lactate, myristyl lactate, oleyl lactate, etc.

Other skin permeation enhancers include hexahydro-1-dodecyl-2H-azepin-2-one (laurocapram, Azone™) and derivatives thereof, dimethylsulfoxide (DMSO), n-decyl methylsulfoxide, salicylic acid and alkyl esters thereof, e.g., methyl salicylate, N,N-dimethylacetamide, dimethylformamide, N,N-dimethyltoluamide, 2-pyrrolidinone and N-alkyl derivatives thereof, e.g., NMP and N-octyl-2-pyrrolidinone, 2-nonyl-1,3-dioxolane, eucalyptol and sorbitan esters.

Other ingredients of the composition can include one or more excipients selected from thickening agents, humectants, fillers, preservatives, cross-linking agents, surfactants, emulsifiers, pH adjusting agents, antioxidants, stabilizers, colors and fragrances. Suitable thickening agents include polyacrylic acid, sodium polyacrylate, carboxymethylcellulose (carmellose) sodium, polyvinyl alcohol, polyvinyl pyrrolidone (PVP), gelatin, etc. Suitable humectants include glycerol, propylene glycol, PEG, 1,3-butanediol and sorbitol. Suitable fillers include kaolin and bentonite. Suitable preservatives include p-benzoic acid esters (parabens) and sorbic acid. A mixture of methylparaben and propylparaben is particularly suitable. Suitable cross-linking agents include polyvalent salts such as aluminum and calcium compounds, for example aluminum chloride, aluminum potassium sulfate, aluminum sulfate, calcium phosphate, aluminum acetate, dihydroxyaluminum aminoacetate, calcium chloride and calcium carbonate. Suitable surfactants include glycerol esters of fatty acids, polyoxyethylene sorbitan fatty acid esters (polysorbates), propylene glycol esters of fatty acids, polyoxyethylene castor oil, etc. Suitable pH adjusting agents include acidifying agents such as organic acids, for example citric acid, fumaric acid, malic acid and tartaric acid. A skin irritation reducing agent, such as vitamin E, glycyrrhetic acid or diphenhydramine, can also be present. In a poultice formulation, the coating layer is typically an aqueous gel and water is a major component.

It has been found advantageous to include PVP in a composition, especially in a tape composition, of the invention. Many of the active agents contemplated herein, including valdecoxib, have a tendency to crystallize out of solution over a period of time, and it has been found that PVP is a very effective crystallization inhibitor. Presence of PVP enables concentration of the active agent to be increased in the composition, leading to enhanced skin permeation.

Illustratively a tape composition of the invention has a coating layer that comprises amounts of various ingredients as follows (all percentages by weight):

| | |
|---|---|
| active agent, e.g., valdecoxib | 0.1-10% |
| solvent system | 0.5-20% |
| crystallization inhibitor, e.g., PVP | 0-30% |
| skin permeation enhancer(s) | 0-20% |
| adhesive system | balance to 100% |

A preferred coating composition for a tape formulation of the invention has the following composition:
valdecoxib, 0.2-7%, more preferably 0.5-5%, e.g., 1-3%
NMP (solvent), 1-20%, more preferably 2-10%, e.g., 3-8%
crotamiton (solvent), 0-10%, more preferably 0-5%, e.g., 0.5-2%
PVP, 0-20%, more preferably 1-10%, e.g., 2-7%
oleic acid (skin permeation enhancer), 0-10%, more preferably 0.5-5%, e.g., 1-3%
adhesive system comprising SIS block copolymer, hydrogenated rosin
   glycerol ester, polybutene, liquid paraffm and BHT, balance to 100% it being understood that substitution of other ingredients having similar properties can be made if desired. Typically the adhesive system in such a preferred coating composition constitutes 80-95% by weight of the coating composition and itself illustratively contains:
SIS block copolymer, 10-25%
hydrogenated rosin glycerol ester or equivalent tackifier, 20-40%
polybutene or equivalent liquid rubber, 5-20%
liquid paraffm or equivalent softening agent, 10-40%
BHT or equivalent antioxidant, 1-40%

Illustratively a poultice composition ofthe invention has a coating layer that comprises amounts of various ingredients as follows (all percentages by weight):

| | |
|---|---|
| active agent, e.g., valdecoxib | 0.1-2% |
| solvent system | 0.5-20% |
| thickener(s) | 0-10% |
| humectant(s) | 0-60% |
| skin permeation enhancer(s) | 0-20% |
| preservative(s) | 0-1% |
| adhesive system | 1-20% |
| water and other optional ingredients | balance to 100% |

A preferred coating composition for a poultice formulation of the invention has the following composition:
valdecoxib, 0.2-1.5%, more preferably 0.3-1%, e.g., 0.4-0.5%
NMP, 1-15%, more preferably 2-10%, e.g., 3-8%
crotamiton, 0.2-10%, more preferably 0.5-5%, e.g., 1-3%
oleic acid, 0-10%, more preferably 0.5-5%, e.g., 1-3%
polyacrylate adhesive, 1-10%, more preferably 1.5-7%, e.g., 2-4% (solids weight, typically provided in aqueous solution)
organic acid, 0-5%, more preferably 0-2%, e.g., 0.2-1%
glycerol, 5-50%, more preferably 10-40%, e.g., 20-30%
sodium polyacrylate, 0-1 5%, more preferably 0-8%, e.g., 2-6%
carmellose sodium, 0-15%, more preferably 0-8%, e.g., 2-6%
hydroxypropylcellulose, 0-10%, more preferably 0-6%, e.g., 1-4%
polyvalent salt, 0-2%, more preferably 0-1%, e.g., 0.05-0.5%
disodium edetate, 0-1%, more preferably 0-0.5%, e.g., 0.02-0.2%
propylene glycol, 0-30%, more preferably 0-20%, e.g., 5-15%
paraben, 0-1%, more preferably 0.05-0.5%, e.g., 0.1-0.3%
castor oil, 0-5%, more preferably 0-2%, e.g., 0.1-1%
surfactant, 0-5%, more preferably 0-2%, e.g., 0.1-1%
urea, 0-10%, more preferably 0-5%, e.g., 0.5-2%
menthol, 0-5%, more preferably 0-2%, e.g., 0.1-1%
water and other optional ingredients, balance to 100% it being understood that substitution of other ingredients having similar properties can be made if desired.

Certain compounds listed above as permeation enhancers can function as topical analgesics in their own right. For example, methyl salicylate, menthol or a combination thereof can provide complementary analgesia when included in a composition of the present invention. In particular, such compounds can provide early-onset, short-term analgesia that complements the longer-term, sustained analgesic and anti-inflammatory effects of the active agent. In compositions of the invention comprising methyl salicylate and menthol, suitable amounts are 5-30% by weight of methyl salicylate and 2-20% by weight of menthol. Amounts outside these ranges can also be useful in particular situations.

Compositions of the invention can be prepared by any known process. Two illustrative processes are described herein as a “mixing process” and a “hot melt process”.

According to the mixing process, which is especially suited to preparation of a poultice, the active agent is first dissolved in the solvent system. Optionally, one or more excipient ingredients other than the adhesive, including for example one or more skin permeation enhancers, are added to the resulting solution, which is mixed thoroughly, with agitation and/or sonication if necessary, to form a premix. Separately, an aqueous gel is prepared by mixing water, adhesive and other water soluble materials as desired, including for example one or more thickening agents and/or humectants. The premix is then added to the gel with thorough mixing. It is usually desirable to conduct this mixing in a way that minimizes air entrapment, for example by kneading, or to remove air from the mixture before proceeding to the next step. The mixture is then coated on a suitable release liner at a desired thickness. A suitable backing sheet is placed over the coating and is pressed to ensure good contact between the coating and the backing sheet. The resulting patch composition can be cut to any desired size and packaged in any suitable packaging, for example a polyethylene or metallic foil pouch.

According to the hot melt process, which is especially suited to preparation of a tape, a pressure-sensitive adhesive composition is first provided. Typically such a composition comprises a thermoplastic polymer system such as natural rubber or a styrenic block copolymer (e.g., SIS), a tackifier resin, a plasticizer and an antioxidant. The adhesive composition is heated with mixing, at a temperature sufficient to melt the adhesive but not so high as to cause significant degradation of the active agent. A solution of the active agent, and optionally other ingredients including one or more skin permeation enhancers, in the solvent system is added to the resulting melted adhesive, with thorough mixing to provide a coating composition, which is then coated on a suitable release liner at a desired thickness. A suitable backing sheet is placed over the coating on the liner and is pressed to ensure good contact between the coating and the backing sheet. The resulting patch composition can be cut and packaged as in the mixing process.

The composition can be designed so that the drug penetrates the skin to deliver a therapeutically effective amount of the drug to a target site such as epidermal, dermal, subcutaneous, muscular and articular organs and tissues while maintaining systemic levels of the drug not greatly in excess of a minimum therapeutically effective level. Thus the present composition can be used to effect targeted delivery of valdecoxib or a prodrug thereof to an external or internal site of pain and/or inflammation in a subject. According to a first therapeutic method of the invention, a composition as provided herein is topically administered to a skin surface of the subject, preferably at a locus overlying or adjacent to the site of pain and/or inflammation.

Compositions as provided herein can alternatively be used to effect systemic treatment of a subject having a COX-2 mediated disorder. According to a second therapeutic method of the invention, a composition as provided herein is administered transdermally, preferably by contacting the composition with an area of skin of the subject not greater than about 400 cm².

Therapeutic methods and compositions of the invention are useful in treatment and prevention of a very wide range of disorders mediated by COX-2, including but not restricted to disorders characterized by inflammation, pain and/or fever. Such compositions are especially useful as anti-inflammatory agents, such as in treatment of arthritis, with the additional benefit of having significantly less harmful side effects, especially when systemically administered, than compositions of conventional NSAIDs that lack selectivity for COX-2 over COX-1. Thus compositions of the invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

Such compositions are useful in treatment of asthma, bronchitis, menstrual cramps, preterm labor, tendinitis, bursitis, allergic neuritis, cytomegalovirus infectivity, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, scleroderma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic diseases, such as retinitis, conjunctivitis, retinopathies, uveitis, ocular photophobia, and of acute injury to the eye tissue.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are used in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, and trauma following surgical and dental procedures.

Such compositions are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful in treatment of angiogenesis-related disorders in a subject, for example to inhibit tumor angiogenesis. Such compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemangiomas, angiofibroma of the nasopharynx and avascular necrosis of bone; and disorders of the female reproductive system such as endometriosis.

Such compositions are useful in the treatment of pre-cancerous diseases, such as actinic keratosis.

Such compositions are useful in prevention, treatment and inhibition of benign and malignant tumors and neoplasia including neoplasia in metastasis, for example in colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such compositions can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in patients at risk of FAP.

More particularly, the compositions can be used in treatment, prevention and inhibition of acral lenuiginous melanoma, actinic keratoses, adenocarcinoma, adenoid cystic carcinoma, adenoma, adenosarcoma, adenosquamous carcinoma, astrocytic tumors, bartholin gland carcinoma, basal cell carcinoma, breast cancer, bronchial gland carcinoma, capillary hemangioma, carcinoids, carcinosarcoma, cavernous hemangioma, cholangiocarcinoma, chondrosarcoma, chorioid plexus papilloma or carcinoma, clear cell carcinoma, cutaneous T-cell lymphoma (mycosis fungoides), cystadenoma, displastic nevi, endodermal sinus tumor, endometrial hyperplasia, endometrial stromal sarcoma, endometrioid adenocarcinoma, ependymoma, epithelioid angiomatosis, Ewing's sarcoma, fibrolamellar sarcoma, focal nodular hyperplasia, gastrinoma, germ cell tumors, glioblastoma, glucagonoma, hemangioblastoma, hemangioendothelioma, hemangioma, hepatic adenoma, hepatic adenomatosis, hepatocellular carcinoma, insulinoma, intraepithelial neoplasia, interepithelial squamous cell neoplasia, invasive squamous cell carcinoma, Kaposi's sarcoma, large cell carcinoma, leiomyosarcoma, lentigo-maligna melanoma, malignant melanoma, malignant mesothelial tumors, medulloblastoma, medulloepithelioma, melanoma, meningioma, mesothelioma, mucoepidermoid carcinoma, neuroblastoma, neuroepithelial adenocarcinoma, nodular melanoma, oat cell carcinoma, oligodendroglioma, osteosarcoma, papillary serous adenocarcinoma, pineal tumors, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinoma, somatostatin-secreting tumor, squamous carcinoma, squamous cell carcinoma, submesothelial carcinoma, superficial spreading melanoma, undifferentiated carcinoma, uveal melanoma, verrucous carcinoma, vipoma, well differentiated carcinoma and Wilm's tumor.

Such compositions inhibit prostanoid-induced smooth muscle contraction by inhibiting synthesis of contractile prostanoids and hence can be of use in treatment of dysmenorrhea, premature labor, asthma and eosinophil-related disorders. They also can be of use for decreasing bone loss particularly in postmenopausal women (i.e., treatment of osteoporosis), and for treatment of glaucoma.

Preferred uses for compositions of the invention are for treatment of rheumatoid arthritis and osteoarthritis, for pain management generally (particularly post-oral surgery pain, post-general surgery pain, post-orthopedic surgery pain, and acute flares of osteoarthritis), for prevention and treatment of headache and migraine, for treatment of Alzheimer's disease, and for colon cancer chemoprevention.

Topical application of a composition of the invention can be especially useful in treatment of any kind of dermal disorder having an inflammatory component, whether malignant, non-malignant or pre-malignant, including scar formation and ketosis, and also including burns and solar damage, for example sunburn, wrinkles, etc. Such compositions can be used to treat inflammation resulting from a variety of skin injuries including without limitation those caused by viral diseases including herpes infections (e.g., cold sores, genital herpes), shingles and chicken pox. Other lesions or injuries to the skin that can be treated with such compositions include pressure sores (decubitus ulcers), hyperproliferative activity in the epidermis, miliria, psoriasis, eczema, acne, dermatitis, itching, warts and rosacea. Such compositions can also facilitate healing processes after surgical procedures, including cosmetic procedures such as chemical peels, laser treatment, dermabrasion, face-lifts, eyelid surgery, etc.

Besides being useful for human treatment, compositions of the invention are also useful for veterinary treatment of companion animals, exotic animals, farm animals, and the like, particularly mammals including rodents. More particularly, compositions-of the invention are useful for veterinary treatment of COX-2 mediated disorders in horses, dogs and cats.

The present compositions can be used in combination therapies with opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise use of a composition of the invention with one or more compounds selected from aceclofenac, acemetacin, ε-acetamidocaproic acid, acetaminophen, acetaminosalol, acetanilide, acetylsalicylsalicylic acid, S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, aspirin, balsalazide, bendazac, benorylate, benoxaprofen, benzpiperylon, benzydamine, benzylmorphine, berberine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butorphanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, dipyrocetyl, dipyrone, ditazol, droxicam, emorfazone, enfenamic acid, epirizole, eptazocine, etanercept, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, infliximab, interleukin-10, isofezolac, isoladol, isomethadone, isonixin, isoxepac, isoxicam, ketobemidone, ketoprofen, ketorolac, p-lactophenetide, lefetamine, levorphanol, lexipafant, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, pirazolac, piritramide, piroxicam, pirprofen, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalate, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiarmide, tilidine, tinoridine, tolfenamic acid, tolmetin, tramadol, tropesin, viminol, xenbucin, ximoprofen, zaltoprofen, ziconotide and zomepirac (see The Merck Index, 13th Edition (2001), Therapeutic Category and Biological Activity Index, lists therein headed “Analgesic”, “Anti-inflammatory” and “Antipyretic”).

Particularly preferred combination therapies comprise use of a composition of the invention with an opioid compound, more particularly where the opioid compound is codeine, meperidine, morphine or a derivative thereof.

The compound to be administered in combination with the composition of the invention can be formulated separately therefrom, and administered by any suitable route, including orally, rectally, parenterally or topically to the skin or elsewhere. Alternatively, the compound to be administered in combination with the present composition can be coformulated therewith as a patch composition.

In an embodiment of the invention, particularly where the COX-2 mediated condition is headache or migraine, the present composition is administered in combination therapy with a vasomodulator, preferably a xanthine derivative having vasomodulatory effect, more preferably an alkylxanthine compound.

Combination therapies wherein an alkylxanthine compound is co-administered with a composition as provided herein are embraced by the present embodiment of the invention whether or not the alkylxanthine is a vasomodulator and whether or not the therapeutic effectiveness of the combination is to any degree attributable to a vasomodulatory effect. The term “alkylxanthine” herein embraces xanthine derivatives having one or more C₁₋₄ alkyl, preferably methyl, substituents, and pharmaceutically acceptable salts of such xanthine derivatives. Dimethylxanthines and trimethylxanthines, including caffeine, theobromine and theophylline, are especially preferred. Most perferably, the alkylxanthine compound is caffeine.

The vasomodulator or alkylxanthine component of the combination therapy can be administered in any suitable dosage form by any suitable route, including orally, rectally, parenterally or topically to the skin or elsewhere. The vasomodulator or alkylzanthine can optionally be coformulated with the present composition in a single transdermal dosage form. Thus a transdermal composition of the invention optionally comprises both valdecoxib or a prodrug thereof or a salt thereof and a vasomodulator or alkylxanthine such as caffeine, in total and relative amounts that are therapeutically effective.

### EXAMPLES

This invention will be more fully described by way of the following Examples but is not limited to these Examples.

### Example 1

In order to identify candidate solvent systems for patch formulations of selective COX-2 inhibitory drugs of low water solubility, various solvents were tested for solubility of celecoxib and valdecoxib at room temperature. Results are shown in Table 1.

TABLE 1
Solubility of celecoxib and valdecoxib in various solvents

solubility (mg/g)

| solvent | celecoxib | valdecoxib |
|---|---|---|
| PEG 400 | 340 | 198 |
| dipropylene glycol | 44 | 67 |
| propylene glycol | 28 | 30 |
| 1,3-butylene glycol | 20 | 19 |
| glycerol | n.d. | 1 |
| DIA | 94 | 20 |
| diethyl sebacate | 77 | 10 |
| crotamiton | 306 | 165 |
| NMP | 219 | 190 |

n.d. = not determined

PEG 400, crotamiton and NMP exhibited the greatest solubility of celecoxib and valdecoxib among the solvents tested.

### Example 2

As a way of measuring the skin permeation properties of selective COX-2 inhibitory drugs by comparison with certain nonselective NSAIDs commonly used in patch formulations, a 10 ml Franz diffusion cell was provided utilizing a rat abdominal skin membrane and a receptor medium of 10% NMP in Dulbecco's phosphate buffer saline (without calcium or magnesium), 1× at pH 7.4. A 15 mm disk of the membrane was placed on a diffusion cell filled with the receptor fluid and the diffusion cell was maintained at 32° C. A 10 mM solution of each drug in NMP was placed in an amount of 1 ml on the membrane. The amount of drug that had permeated through the membrane by various times in an 8-10 hour period was determined by HPLC analysis of the receptor fluid. The test was conducted in 3 replicates. Skin flux data were calculated and results are shown in Table 2.

TABLE 2
Skin flux of celecoxib, valdecoxib and commonly used NSAIDs

| drug | skin flux (nmol/cm² · h) |
|---|---|
| celecoxib | 1.6 |
| valdecoxib | 3.3 |
| felbinac | 64.6 |
| ketoprofen | 33.4 |

Skin flux of the selective COX-2 inhibitory drugs celecoxib and valdecoxib was found to be lower by at least an order of magnitude than that of the NSAIDs felbinac and ketoprofen. This illustrates the technical difficulty of providing an effective patch formulation of a selective COX-2 inhibitory drug of low water solubility.

### Example 3

An in vitro skin permeation study was conducted by a procedure similar to that of Example 2, but using Dulbecco's phosphate buffer saline (without calcium or magnesium), 1× as the receptor medium. The test solutions in this example comprised celecoxib or valdecoxib at a concentration of 1% weight/volume in various solvents. The test was conducted in 3 replicates. Skin flux data were calculated and results are shown in Table 3.

TABLE 3
Skin flux of celecoxib and valdecoxib in various solvents

skin flux (μg/cm² · h)

| solvent | celecoxib | valdecoxib |
|---|---|---|
| NMP | 0.15 | 0.51 |
| PEG 400 | not detectable | not detectable |
| crotamiton | not detectable | 0.02 |

Skin flux was much higher when either celecoxib or valdecoxib was dissolved in NMP than in PEG 400 or crotamiton.

### Example 4

An in vitro skin permeation study was conducted by the same procedure as that of Example 3. The test solutions in this example comprised celecoxib or valdecoxib at a concentration of 1% weight/volume in NMP, with or without oleic acid at 1% weight/volume. The test was conducted in 3 replicates. Skin permeation results are shown in FIGS. 3 (celecoxib) and 4 (valdecoxib).

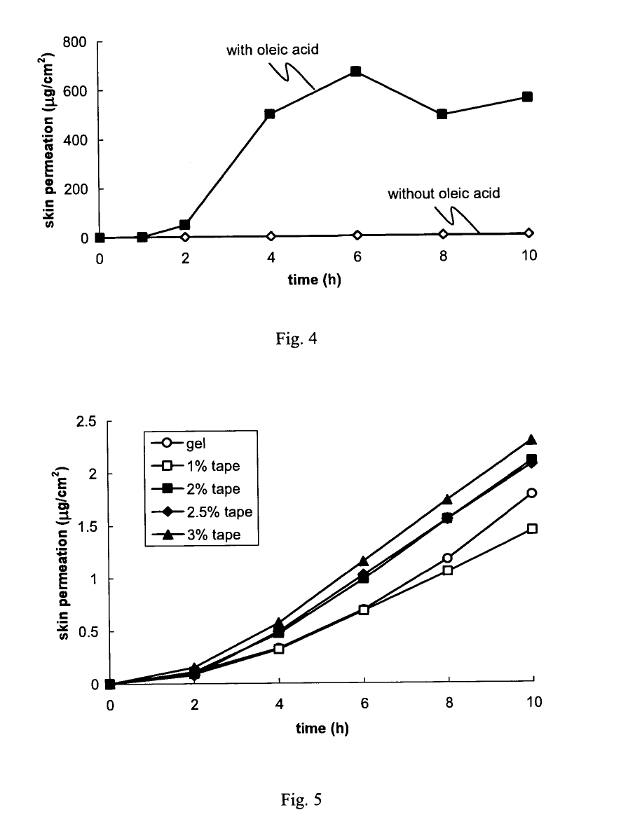

For both selective COX-2 inhibitory drugs in NMP solution, oleic acid was found to strongly enhance skin permeation. In the case of valdecoxib (FIG. 4), amount of drug permeated reached a plateau after about 4 hours, probably as a result of saturation of the receptor medium.

### Example 5

Poultice formulations were prepared containing 0.5% by weight celecoxib or valdecoxib, using PEG 400 and crotamiton as solvents. The poultice formulations were prepared by a method substantially as described in Example 29 below. Composition by weight of the poultice formulations was:

| | |
|---|---|
| celecoxib or valdecoxib | 0.5% |
| crotamiton | 1.0% |
| PEG 400 | 15.0% |
| oleic acid | 1.0% |
| polyacrylate adhesive, 20% aqueous solution | 10.0% |
| organic acid | 0.5% |
| glycerol | 25.5% |
| sodium polyacrylate | 6.0% |
| carmellose sodium | 4.0% |
| hydroxypropylcellulose | 1.0% |
| polyvalent salt | 0.1% |
| disodium edetate | 0.05% |
| paraben | 0.15% |
| castor oil | 0.5% |
| surfactant | 0.5% |
| purified water | q.s. to 100% |

A 25 mm disk was punched from each poultice and placed on a Franz diffusion cell. A skin permeation study was conducted according to the procedure of Example 3. For comparison, a gel composition of each drug was also tested, in an amount of 200 mg. Composition by weight of the gel composition was:

| | |
|---|---|
| celecoxib or valdecoxib | 1.0% |
| hydroxypropylcellulose | 2.5% |
| ethanol | 70.0% |
| water | 26.5% |

The test was conducted in 3 replicates. Skin flux data were calculated and results are shown in Table 4.

TABLE 4
Skin flux of celecoxib and valdecoxib poultices and gels

skin flux (μg/cm² · h)

| formulation | celecoxib | valdecoxib |
|---|---|---|
| 0.5% poultice | 0.008 | 0.009 |
| 1.0% gel | 0.039 | 0.115 |

The poultice formulations exhibited much lower skin flux than the gel formulations, even when the lower concentration of drug in the poultice formulations was taken into account. This further illustrates the technical difficulty of formulating a selective COX-2 inhibitory drug of low water solubility as a patch, especially where a hydrophilic poultice system is desired.

### Example 6

Tape formulations were prepared containing valdecoxib, using NMP or crotamiton as solvents. The tape with NMP contained 1% valdecoxib and the tape with crotamiton contained 2% valdecoxib. The tape formulations also contained 1% by weight oleic acid. They were prepared by a method substantially as described in Example 30 below. Composition by weight of the 1% valdecoxib tape was:

| | |
|---|---|
| valdecoxib | 1% |
| NMP | 5% |
| PEG 400 | 2% |
| oleic acid | 1% |
| SIS copolymer | 15% |
| hydrogenated rosin glycerol ester | 30% |
| polybutene | 10% |
| liquid paraffin | 34% |
| BHT | 2% |

and composition by weight of the 2% valdecoxib tape was:

| | |
|---|---|
| valdecoxib | 2% |
| crotamiton | 2% |
| PEG 400 | 5% |
| oleic acid | 1% |
| SIS copolymer | 15% |
| hydrogenated rosin glycerol ester | 30% |
| polybutene | 10% |
| liquid paraffin | 33% |
| BHT | 2% |

A skin permeation study was conducted exactly as in Example 5, by comparison with the valdecoxib gel formulation described in Example 5. The test was conducted in 3 replicates. Skin flux data were calculated and results are shown in Table 5.

TABLE 5
Skin flux of valdecoxib tapes and gel

| formulation | skin flux (μg/cm² · h) |
|---|---|
| 1% valdecoxib tape, NMP solvent | 0.069 |
| 2% valdecoxib tape, crotamiton solvent | 0.016 |
| 1% valdecoxib gel | 0.090 |

Valdecoxib skin flux from the tape having NMP as the solvent was comparable to slightly lower than that from the gel. The tape containing 2% valdecoxib together with crotamiton as the solvent exhibited much lower skin flux, in spite of the higher concentration of valdecoxib in the tape.

### Example 7

A tape formulation of celecoxib, having crotamiton as the solvent, was tested for anti-inflammatory activity in a carrageenan-induced paw edema assay in rats. This assay provides a pharmacological model for acute inflammation. For comparison, the 1% celecoxib gel formulation described in Example 5 was also tested. The tape formulation was prepared by a method substantially as described in Example 30 below. Composition by weight of the tape was:

| | |
|---|---|
| celecoxib | 1% |
| crotamiton | 2% |
| PEG 400 | 5% |
| oleic acid | 1% |
| SIS copolymer | 15% |
| hydrogenated rosin glycerol ester | 30% |
| polybutene | 10% |
| liquid paraffin | 34% |
| BHT | 2% |

A group of 8 rats was assigned to each treatment. Volume of the right hind paw of each animal was measured prior to treatment. A test formulation was then applied to the right hind paw and left in position for 4 hours. A control set of rats received no such application. The tape formulation was applied as a 3 cm×4 cm patch. The gel formulation was applied in an amount of 200 mg and covered with plastic wrap. After 4 hours, the formulation was removed and immediately a 1% carrageenan suspension in saline was injected subcutaneously into the planta of the right hind paw. Volume of the right hind paw was measured 2, 3 and 4 hours after carrageenan injection. Swelling rate was calculated by the following equation:

$$swelling rate ( \% ) = 100 \times \frac{V - {V}_{0}}{{V}_{0}}$$

where V₀ is the initial paw volume and V is the paw volume 2, 3 or 4 hours after carrageenan injection. Data are shown in Table 6.

TABLE 6
Inhibition of carrageenan-induced paw edema

| | swelling rate (%) | | |
|---|---|---|---|
| treatment | 2 h | 3 h | 4 h |
| no application | 85.6 ± 3.3 | 93.7 ± 4.1 | 92.6 ± 4.5 |
| 1% celecoxib gel | 54.0 ± 3.6 | 58.9 ± 3.0 | 63.7 ± 3.0 |
| | (37) | (37) | (31) |
| 1% celecoxib tape | 68.4 ± 3.2 | 72.5 ± 4.4 | 72.7 ± 3.7 |
| (crotamiton solvent) | (20) | (23) | (22) |

( ) % inhibition of swelling

The celecoxib tape of this example was less effective in reducing swelling in the carrageenan-induced paw edema assay than the celecoxib gel.

### Example 8

Two tape formulations of valdecoxib, having NMP or crotamiton as the solvent, were tested for anti-inflammatory activity in a carrageenan-induced paw edema assay in rats, by the procedure described in Example 7. The tape formulations were those described in Example 6. For comparison, the 1% valdecoxib gel formulation described in Example 5 was also tested. Data are shown in Table 7.

TABLE 7
Inhibition of carrageenan-induced paw edema

| | swelling rate (%) | | |
|---|---|---|---|
| treatment | 2 h | 3 h | 4 h |
| no application | 63.6 ± 3.2 | 70.9 ± 2.3 | 81.4 ± 2.9 |
| 1% valdecoxib gel | 35.2 ± 3.9 | 44.1 ± 3.7 | 54.8 ± 3.1 |
| | (45) | (38) | (33) |
| 1% valdecoxib tape | 58.5 ± 3.7 | 61.8 ± 3.6 | 59.9 ± 3.8 |
| (NMP solvent) | (8) | (13) | (26) |
| 2% valdecoxib tape | 61.9 ± 2.7 | 63.3 ± 1.7 | 65.9 ± 2.7 |
| (crotamiton solvent) | (3) | (11) | (19) |

( ) % inhibition of swelling

The valdecoxib tapes of this example were less effective in reducing swelling in the carrageenan-induced paw edema assay than the valdecoxib gel.

### Example 9

A modified carrageenan-induced paw edema assay was conducted using the 1% celecoxib and valdecoxib gel and tape formulations of Examples 7 and 8. The procedure was as described in Example 7, except that the application of gel or tape was made to the back rather than to the right hind paw of each animal. Data are shown in Table 8.

TABLE 8
Inhibition of carrageenan-induced paw edema (modified assay)

| | swelling rate (%) | | |
|---|---|---|---|
| treatment | 2 h | 3 h | 4 h |
| no application | 71.9 ± 3.9 | 75.9 ± 3.4 | 80.5 ± 4.0 |
| 1% celecoxib gel | 22.6 ± 3.0 | 37.1 ± 4.3 | 40.6 ± 4.8 |
| | (69) | (51) | (50) |
| 1% celecoxib tape | 53.5 ± 5.5 | 61.1 ± 5.4 | 61.9 ± 5.7 |
| (crotamiton solvent) | (26) | (19) | (23) |
| 1% valdecoxib gel | 25.5 ± 2.2 | 35.4 ± 4.9 | 40.3 ± 5.2 |
| | (65) | (53) | (50) |
| 1% valdecoxib tape | 26.1 ± 3.1 | 33.2 ± 3.8 | 42.1 ± 4.5 |
| (NMP solvent) | (64) | (56) | (48) |

( ) % inhibition of swelling

In this modified assay, the valdecoxib tape was equal to the valdecoxib gel in reducing swelling. The celecoxib tape was still inferior to the celecoxib gel in inhibition of swelling in this assay.

### Example 10

The 1% celecoxib and valdecoxib tapes tested in Example 9 were further tested for primary skin irritation by application to normal and abraded skin of Japanese White rabbits. Placebo tapes, having the same composition but lacking only the active agent, were tested for comparison. Primary irritation index (PII) according to Draize criteria was as shown in Table 9. Note that a PII <2 defines “mild irritation” according to the criteria.

TABLE 9
Primary skin irritation of tape formulations

| formulation | PII |
|---|---|
| (A) 1% celecoxib tape, crotamiton solvent | 0.6 |
| placebo for (A) | 0.6 |
| (B) 1% valdecoxib tape, NMP solvent | 0.9 |
| placebo for (B) | 1.0 |

The tape formulations of the invention exhibited mild irritation, no greater than the placebo tapes.

### Example 11

The modified carrageenan-induced paw edema assay was used to compare anti-inflammatory activity in this acute inflammation model of the following formulations:
0.5% celecoxib poultice, crotamiton/PEG 400 solvent system
2% celecoxib tape, crotamiton/PEG 400 solvent system
0.5% valdecoxib poultice, crotamiton/PEG 400 solvent system
2% valdecoxib tape, crotamiton/PEG 400 solvent system
1% valdecoxib tape, NMP/PEG 400 solvent system
   The poultice formulations were those described in Example 5. The valdecoxib tape formulations were those described in Example 6. Composition of the 2% celecoxib tape was identical to that of the 2% valdecoxib tape except for the active agent. Also included for comparison was a 2% ketoprofen tape available commercially in Japan (Mohrus™ tape).

Data showing percent inhibition of swelling 3 hours after injection in the modified assay (patches applied to the back of the animal) are presented in Table 10.

TABLE 10
Inhibition of carrageenan-induced paw edema (modified assay)

| formulation | % inhibition of swelling |
|---|---|
| 0.5% celecoxib poultice | 23 |
| 2% celecoxib tape | −6 |
| 0.5% valdecoxib poultice | 40 |
| 2% valdecoxib tape (crotamiton/PEG 400) | 39 |
| 1% valdecoxib tape (NMP/PEG 400) | 55 |
| 2% ketoprofen tape | 74 |

The 2% celecoxib tape formulation of this example did not reduce swelling in this assay, but all other compositions exhibited some reduction of swelling. The 1% valdecoxib tape containing NMP came closest to matching the anti-inflammatory efficacy of the commercial 2% ketoprofen tape.

### Example 12

Solubility of valdecoxib in various NMP/PEG 400 mixtures at room temperature was determined in an effort to identify a superior solvent system for use in a valdecoxib patch. Solubility was determined by preparing a saturated solution of valdecoxib in the test solvent system at 80° C. and cooling this solution for 24 hours at room temperature.

Table 11 shows solubility of valdecoxib in various NMP/PEG 400 mixtures, and also shows the concentration of valdecoxib achieved in a poultice formulation similar to that described in Example 5.

TABLE 11
Solubility and formulation concentration of valdecoxib

| NMP:PEG 400 ratio | solubility (mg/g) |
|---|---|
| 0:5 | 169 |
| 5:0 | 190 |
| 5:1 | 149 |
| 5:2 | 126 |
| 5:3 | 100 |
| 5:4 | 73 |
| 5:5 | 71 |

Highest solubility in an NMP-based solvent was obtained in the absence of PEG 400.

### Example 13

The effect of varying oleic acid content from 0.5% to 2% on skin permeation of a valdecoxib tape formulation was investigated in an in vitro study, by the procedure described in Example 5. Composition by weight of the tape formulations was:

| | | | | |
|---|---|---|---|---|
| valdecoxib | 1% | 1% | 1% | 1% |
| NMP | 5% | 5% | 5% | 5% |
| PEG 400 | 2% | 2% | 2% | 2% |
| oleic acid | 0.5% | 1% | 1.5% | 2% |
| SIS copolymer | 15% | 15% | 15% | 15% |
| hydrogenated rosin glycerol ester | 30% | 30% | 30% | 30% |
| polybutene | 10% | 10% | 10% | 10% |
| liquid paraffin | 34.5% | 34% | 33.5% | 33% |
| BHT | 2% | 2% | 2% | 2% |

Skin flux data were calculated and are presented in Table 12.

TABLE 12
Skin flux of valdecoxib tapes

| oleic acid (%) | skin flux (μg/cm² · h) |
|---|---|
| 0.5 | 0.154 |
| 1.0 | 0.166 |
| 1.5 | 0.155 |
| 2.0 | 0.165 |

No response of skin flux to increasing oleic acid concentration was observed.

### Example 14

The effect of addition to a valdecoxib tape formulation of three skin permeation enhancer candidates was investigated in an in vitro study, by the procedure described in Example 5. Each of N-octyl-2-pyrrolidone, N-dodecyl-2-pyrrolidone and cetyl lactate was added at 1% to the valdecoxib tape formulation. Composition by weight of the tape formulations was:

| | | | | |
|---|---|---|---|---|
| valdecoxib | 1% | 1% | 1% | 1% |
| NMP | 5% | 5% | 5% | 5% |
| PEG 400 | 2% | 2% | 2% | 2% |
| oleic acid | 1% | 1% | 1% | 1% |
| SIS copolymer | 15% | 15% | 15% | 15% |
| hydrogenated rosin glycerol ester | 30% | 30% | 30% | 30% |
| polybutene | 10% | 10% | 10% | 10% |
| liquid paraffin | 34% | 33% | 33% | 33% |
| N-octyl-2-pyrrolidone | 0% | 1% | 0% | 0% |
| N-dodecyl-2-pyrrolidone | 0% | 0% | 1% | 0% |
| cetyl lactate | 0% | 0% | 0% | 1% |
| BHT | 2% | 2% | 2% | 2% |

Skin flux data were calculated and are presented in Table 13.

TABLE 13
Skin flux of valdecoxib tapes

| enhancer candidate | skin flux (μg/cm² · h) |
|---|---|
| no enhancer | 0.207 |
| 1% N-octyl-2-pyrrolidone | 0.219 |
| 1% N-dodecyl-2-pyrrolidone | 0.200 |
| 1% cetyl lactate | 0.216 |

No response of skin flux was observed to presence of any of the skin permeation enhancer candidates tested in this example.

### Example 15

The effect of addition to a valdecoxib tape formulation of three additional skin permeation enhancer candidates was investigated in an in vitro study, by the procedure described in Example 5. Each of DIA, diethyl sebacate and isopropyl myristate was added at 3% to the valdecoxib tape formulation. Skin flux data were calculated and are presented in Table 14.

TABLE 14
Skin flux of valdecoxib tapes

| enhancer candidate | skin flux (μg/cm² · h) |
|---|---|
| no enhancer | 0.164 |
| 3% DIA | 0.166 |
| 3% diethyl sebacate | 0.221 |
| 3% isopropyl myristate | 0.233 |

Diethyl sebacate and isopropyl myristate increased valdecoxib skin flux. The formulation with isopropyl myristate did not maintain its relatively high skin flux upon storage for 10 days at 4° C., for reasons that have not been determined.

### Example 16

Efforts to increase valdecoxib concentration in a tape formulation from 1% to 2% or higher, with an NMP/PEG 400 solvent system, led to crystallization of valdecoxib upon storage of the tape in refrigerated conditions. It was found that addition of 5% PVP to a composition having 2% valdecoxib, 5% NMP and either 2% or zero PEG 400 inhibited crystal formation.

Accordingly, a new series of tape formulations were prepared, each having 8% NMP, zero PEG 400, 1% oleic acid and 5% PVP as a solvent system for valdecoxib ranging in concentration from 1% to 3%. An in vitro skin permeation study by the procedure described in Example 5 was conducted on these formulations. For comparison, the 1% valdecoxib gel formulation described in Example 5 was also tested. Results are shown in FIG. 5.

Significantly enhanced skin permeation was achieved with the formulations having higher than 1% valdecoxib concentration; however, differences among formulations having 2%, 2.5% and 3% valdecoxib were small.

Refrigerated storage of these formulations did not lead to reduction in skin permeation.

### Example 17

A 2% valdecoxib tape formulation was compared with three modified formulations in an in vitro skin permeation study by the procedure described in Example 5. The modified formulations had addition of 1%, 3% and 5% PVP respectively. Composition by weight of the tape formulations was:

| | | | | |
|---|---|---|---|---|
| valdecoxib | 2% | 2% | 2% | 2% |
| NMP | 5% | 5% | 5% | 5% |
| oleic acid | 1% | 1% | 1% | 1% |
| PVP | 0% | 1% | 3% | 5% |
| SIS copolymer | 15% | 15% | 15% | 15% |
| hydrogenated rosin glycerol ester | 30% | 30% | 30% | 30% |
| polybutene | 10% | 10% | 10% | 10% |
| liquid paraffin | 35% | 34% | 32% | 30% |
| N-octyl-2-pyrrolidone | 0% | 1% | 0% | 0% |
| N-dodecyl-2-pyrrolidone | 0% | 0% | 1% | 0% |
| cetyl lactate | 0% | 0% | 0% | 1% |
| BHT | 2% | 2% | 2% | 2% |

Skin flux was calculated and results are shown in Table 15.

TABLE 15
Skin flux of valdecoxib tapes

| tape formulation | skin flux (μg/cm² · h) |
|---|---|
| 2% valdecoxib, no PVP | 0.014 |
| 2% valdecoxib, 1% PVP | 0.016 |
| 2% valdecoxib, 3% PVP | 0.090 |
| 2% valdecoxib, 5% PVP | 0.148 |

Significant enhancement of skin flux was obtained with addition of 3% and 5%, but not 1%, PVP.

### Example 18

Valdecoxib tape formulations were prepared having increased coating thickness (400 g/m² instead of the usual 200 g/m²). The tape formulations were prepared by a method substantially as described in Example 30 below. Composition by weight of the tape formulations was:

| | | | | |
|---|---|---|---|---|
| valdecoxib | 1% | 1.5% | 1.5% | % |
| NMP | 5% | 8% | 8% | 8% |
| PEG 400 | 2% | 0% | 0% | 0% |
| oleic acid | 1% | 1% | 1% | 1% |
| PVP | 0% | 5% | 5% | 5% |
| SIS copolymer | 15% | 15% | 15% | 15% |
| hydrogenated rosin glycerol ester | 30% | 30% | 30% | 30% |
| polybutene | 10% | 10% | 10% | 10% |
| liquid paraffin | 34% | 27.5% | 27.5% | 26% |
| N-octyl-2-pyrrolidone | 0% | 1% | 0% | 0% |
| N-dodecyl-2-pyrrolidone | 0% | 0% | 1% | 0% |
| cetyl lactate | 0% | 0% | 0% | 1% |
| BHT | 2% | 2% | 2% | 2% |
| thickness (g/m²) | 200 | 200 | 400 | 400 |

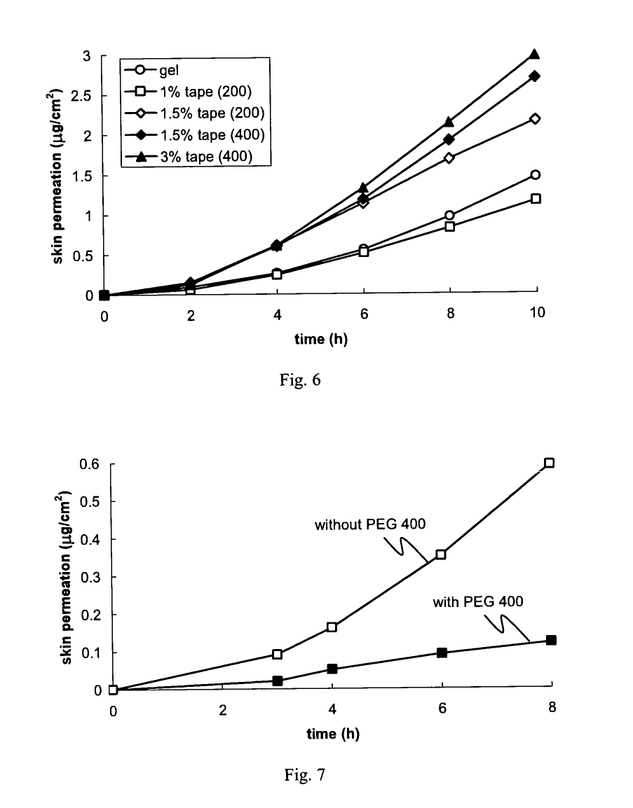

An in vitro skin permeation study by the procedure described in Example 5 was conducted on these formulations. For comparison, the 1% valdecoxib gel formulation described in Example 5 was also tested. Results are shown in FIG. 6.

Effect of increased coating thickness had little effect on skin permeation up to about 4 hours from initiation of the test, but later some further increase in skin permeation was observed.

### Example 19

The valdecoxib tape formulations of Example 18 were placed in a carrageenan-induced paw edema assay as described in Example 7, except that the tape was left in place on the right hind paw for 4 hours or 8 hours prior to injection of carrageenan. For comparison purposes, a placebo tape and a commercial 2% ketoprofen tape (Mohrus tape) were included in the study. Swelling was measured 3 hours after injection. Percentage inhibition of swelling is shown in Table 16.

TABLE 16
Inhibition of carrageenan-induced paw edema

| | % inhibition of swelling | |
|---|---|---|
| tape formulation | 4 h pretreatment | 8 h pretreatment |
| 0% valdecoxib (placebo, 200 g/m²) | −9 | 5 |
| 1.5% valdecoxib (200 g/m²) | 8 | 17 |
| 1.5% valdecoxib (400 g/m²) | 19 | 31 |
| 3% valdecoxib (400 g/m²) | 24 | 32 |
| 2% ketoprofen | 39 | 58 |

### Example 20

Tape formulations containing 1% and 2% valdecoxib were placed in a carrageenan-induced paw edema assay as described in Example 19, by comparison with a placebo tape and a commercial 2% ketoprofen tape (Mohrus tape). Percentage inhibition of swelling following 4 hours and 8 hours pretreatment is shown in Table 17.

TABLE 17
Inhibition of carrageenan-induced paw edema

% inhibition of swelling

| tape formulation | 4 h pretreatment | 8 h pretreatment |
|---|---|---|
| placebo | 3 | 11 |
| 1% valdecoxib | 8 | 17 |
| 2% valdecoxib | 15 | 22 |
| 2% ketoprofen | 43 | 38 |

### Example 21

The 1% valdecoxib tape formulation of Example 18 was tested for anti-inflammatory activity in an adjuvant-induced polyarthritis assay in rats. This assay provides a pharmacological model for chronic inflammation. For comparison, a placebo tape (wherein valdecoxib was substituted by an additional 1% liquid paraffm) and a commercial 2% ketoprofen tape (Mohrus tape) were also tested.

A group of 7 rats was assigned to each treatment. Volume of the right hind paw of each animal was measured prior to treatment. An adjuvant comprising killed bacteria (Mycobacterium butyricum) was injected subcutaneously into the planta of the left hind paw. Fourteen days later, the volume of the right hind paw was measured again, immediately prior to commencement of treatment with the test formulations. Each tape formulation was applied as a 4 cm×4 cm patch to the right (non-injected) hind paw for a period of 6 hours, daily for 8 days. On the 4th, 6th and 8th days after commencement of treatment the volume of the right (non-injected) hind paw was measured again. Swelling rate was calculated as for the carrageenan-induced paw edema assay (Example 7). Data are shown in Table 18.

TABLE 18
Inhibition of adjuvant-induced polyarthritis

| | swelling rate (%) | | | |
|---|---|---|---|---|
| tape formulation | day 0 | day 4 | day 6 | day 8 |
| placebo | 78.9 ± 7.7 | 83.7 ± 8.5 | 89.3 ± 7.8 | 84.1 ± 11.0 |
| 1% valdecoxib | 80.7 ± 8.2 | 50.1 ± 5.8 | 42.6 ± 4.1 | 36.5 ± 5.3 |
| | | (40) | (52) | (57) |
| 2% ketoprofen | 83.6 ± 7.6 | 52.5 ± 5.0 | 40.2 ± 4.2 | 37.1 ± 3.4 |
| | | (37) | (55) | (56) |

( ) % inhibition of swelling versus placebo

Surprisingly, in this model for chronic inflammation, the 1% valdecoxib tape of the invention performed equally to the 2% ketoprofen comparative standard.

### Example 22

The 2% valdecoxib tape tested in Example 20 was further tested for primary skin irritation by application to normal and abraded skin of Japanese White rabbits, by comparison with a placebo tape. Primary irritation index (PII) according to Draize criteria was as shown in Table 19. Note that a PII <2 defines “mild irritation” according to the criteria.

TABLE 19
Primary skin irritation of tape formulations

| formulation | PII |
|---|---|
| (A) 2% valdecoxib tape | 0.4 |
| placebo for (A) | 0.8 |

The 2% valdecoxib tape of the invention exhibited mild irritation, no greater than the placebo tape.

### Example 23

The 0.5% valdecoxib poultice formulation tested in Examples 5 and 11, having a solvent system consisting of 1% crotamiton and 15% PEG 400, was fuirther tested for in vitro skin permeation by the procedure described in Example 5. Also tested was a similar poultice formulation having no PEG 400, and prepared by a similar method. Skin permeation data are shown in FIG. 7.

Removal of PEG 400 greatly enhanced skin permeation in this study.

### Example 24

A 0.5% valdecoxib poultice formulation having a solvent system comprising 2% crotamiton and 5% NMP but no PEG 400 was tested for anti-inflammatory activity in an adjuvant-induced polyarthritis assay in rats, following the procedure described in Example 21. Composition by weight of the poultice formulation was:

| | |
|---|---|
| valdecoxib | 0.5% |
| crotamiton | 2.0% |
| NMP | 5.0% |
| oleic acid | 1.0% |
| polyacrylate adhesive, 20% aqueous solution | 10.0% |
| organic acid | 0.5% |
| glycerol | 25.5% |
| sodium polyacrylate | 6.0% |
| carmellose sodium | 5.0% |
| hydroxypropylcellulose | 2.0% |
| polyvalent salt | 0.1% |
| disodium edetate | 0.04% |
| propylene glycol | 10.0% |
| paraben | 0.15% |
| castor oil | 0.5% |
| surfactant | 0.5% |
| urea | 1.0% |
| purified water | q.s. to 100% |

For comparison, a placebo poultice and a commercial 2% ketoprofen tape (Mohrus tape) were also tested. Data are shown in Table 20.

TABLE 20
Inhibition of adjuvant-induced polyarthritis

| | swelling rate (%) | | | |
|---|---|---|---|---|
| formulation | day 0 | day 4 | day 6 | day 8 |
| placebo poultice | 41.8 ± 2.4 | 85.4 ± 8.3 | 74.2 ± 7.8 | 63.6 ± 7.4 |
| 0.5% valdecoxib poultice | 41.8 ± 2.3 | 42.8 ± 2.8 | 37.5 ± 3.6 | 30.1 ± 2.7 |
| | | (50) | (49) | (53) |
| 2% ketoprofen | 41.4 ± 2.1 | 33.7 ± 3.1 | 22.5 ± 3.9 | 16.4 ± 3.4 |
| | | (61) | (70) | (74) |

( ) % inhibition of swelling versus placebo

### Example 25

A 0.5% valdecoxib poultice formulation having a solvent system comprising 2% crotamiton and 5% NMP but no PEG 400 was tested for in vitro skin permeation by the procedure described in Example 5. Also tested were 0.4% and 0.3% valdecoxib poultice formulations having no PEG 400, but with addition of 1% urea, prepared by a similar method. Composition by weight of the poultice formulations was:

| | | | |
|---|---|---|---|
| valdecoxib | 0.5% | 0.4% | 0.3% |
| crotamiton | 2.0% | 2.0% | 2.0% |
| NMP | 5.0% | 5.0% | 5.0% |
| oleic acid | 1.0% | 1.0% | 1.0% |
| polyacrylate adhesive, 20% aqueous solution | 10.0% | 10.0% | 10.0% |
| organic acid | 0.5% | 0.5% | 0.5% |
| glycerol | 25.5% | 25.5% | 25.5% |
| sodium polyacrylate | 6.0% | 6.0% | 6.0% |
| carmellose sodium | 5.0% | 5.0% | 5.0% |
| hydroxypropylcellulose | 2.0% | 2.0% | 2.0% |
| polyvalent salt | 0.1% | 0.1% | 0.1% |
| disodium edetate | 0.04% | 0.04% | 0.04% |
| propylene glycol | 10.0% | 10.0% | 10.0% |
| paraben | 0.15% | 0.15% | 0.15% |
| castor oil | 0.5% | 0.5% | 0.5% |
| surfactant | 0.5% | 0.5% | 0.5% |
| urea | 0% | 1.0% | 1.0% |

| | |
|---|---|
| purified water | q.s. to 100% |

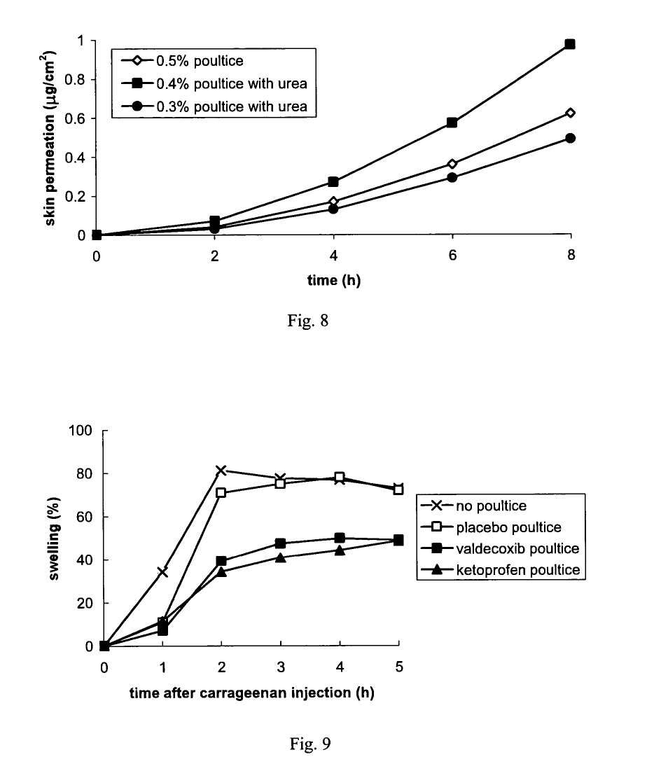

Skin permeation data are shown in FIG. 8. Surprisingly, the poultice formulation with valdecoxib concentration reduced to 0.4%, but with 1% urea added, exhibited enhanced skin permeation in this study.

### Example 26

The 0.4% valdecoxib poultice tested in Example 25 was further tested for primary skin irritation by application to normal and abraded skin of Japanese White rabbits, by comparison with a placebo poultice. Primary irritation index (PII) according to Draize criteria was as shown in Table 21. Note that a PII <2 defines “mild irritation” according to the criteria.

TABLE 21
Primary skin irritation of tape formulations

| formulation | PII |
|---|---|
| (A) 0.4% valdecoxib poultice | 0.8 |
| placebo for (A) | 0.5 |

The 0.4% valdecoxib poultice ofthe invention exhibited mild irritation, comparable to the placebo poultice.

### Example 27

The 0.4% valdecoxib poultice tested in Example 25 was further tested in a modified carrageenan-induced paw edema assay. The poultice was applied as a 3 cm×4 cm patch to the right hind paw and left in place for 1 hour. The patch was removed and a new patch was applied to the same area and left in place for 1 hour. This second patch was removed and carrageenan suspension was then injected into the right hind paw. After injection of carrageenan, yet another new patch was applied to the right (injected) hind paw, and left in place for 1 hour. Swelling rate was determined hourly from 1 to 5 hours after injection. For comparison, a placebo poultice and a 0.3% ketoprofen poultice (Mohrus poultice) were also tested. Results are shown in FIG. 9.

The 0.4% valdecoxib poultice of the invention exhibited comparable anti-inflammatory activity to the 0.3% ketoprofen poultice in this study.

### Example 28

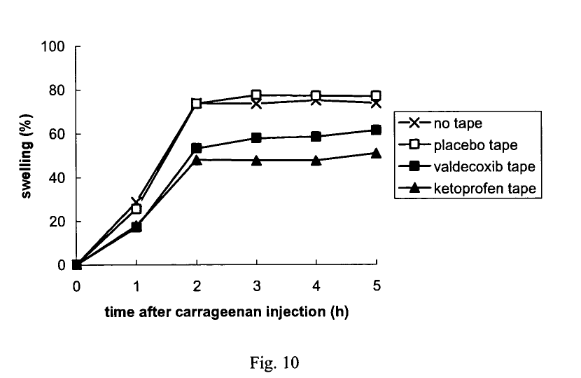

The 2% valdecoxib tape tested in Example 20 was further tested in a modified carrageenan-induced paw edema assay as described in Example 27. For comparison, a placebo tape and a 2% ketoprofen tape (Mohrus tape) were also tested. Results are shown in FIG. 10.

The 2% valdecoxib tape of the invention exhibited anti-inflammatory activity only slightly weaker than the 2% ketoprofen tape in this study.

### Example 29

A poultice formulation of the invention was prepared having the following coating composition (all percentages by weight):

| | |
|---|---|
| valdecoxib | 0.4% |
| NMP | 5.0% |
| crotamiton | 2.0% |
| oleic acid | 1.0% |
| polyacrylate adhesive, 20% aqueous solution | 15.0% |
| organic acid | 0.5% |
| glycerol | 30.0% |
| sodium polyacrylate | 4.5% |
| carmellose sodium | 4.0% |
| hydroxypropylcellulose | 2.0% |
| polyvalent salt | 0.1% |
| disodium edetate | 0.05% |
| propylene glycol | 10.0% |
| paraben | 0.15% |
| castor oil | 0.5% |
| surfactant | 0.5% |
| urea | 1.0% |
| 1 -menthol | 0.5% |
| purified water | q.s. to 100% |

To the glycerol were added the sodium polyacrylate, carmellose sodium, hydroxypropylcellulose and polyvalent salt with mixing until a solution was formed. To this solution were added a portion (about 10% by weight of the fmished coating composition) of the purified water, together with the organic acid, urea, paraben, propylene glycol, disodium edetate, polyacrylate solution, castor oil, surfactant and 1-menthol. The resulting mixture was kneaded for 10 minutes at 35-45° C. to obtain an aqueous gel. Separately, the valdecoxib was dispersed in a mixture of the NMP, crotamiton and oleic acid. The resulting premix was added, together with the remainder of the purified water, to the aqueous gel, which was then kneaded for a further 5 minutes at 35-45° C.

The resulting coating composition was spread over a nonwoven fabric to a thickness of 1000 g/m², and a polypropylene film release liner was laminated over the coating.

### Example 30

A tape formulation of the invention was prepared having the following coating composition (all percentages by weight):

| | |
|---|---|
| valdecoxib | 2% |
| NMP | 5% |
| crotamiton | 1% |
| oleic acid | 1% |
| PVP | 5% |
| SIS copolymer | 15% |
| hydrogenated rosin glycerol ester | 30% |
| polybutene | 10% |
| liquid paraffin | 29% |
| BHT | 2% |

The components of the adhesive system, i.e., SIS copolymer, hydrogenated rosin glycerol ester, polybutene, liquid paraffin and BHT, were blended and then kneaded under a nitrogen stream at 150-200° C. for 60 minutes to form an adhesive melt. Separately, a premix of the NMP, crotamiton, oleic acid, PVP and valdecoxib was prepared, and this premix was then added to the adhesive melt, followed by mixing for 20 minutes.

The resulting coating composition was spread over a liner to a thickness of 200 g/m², and a backing sheet was added.

1. A pharmaceutical composition for application to an area of skin of a subject for local and/or systemic treatment of a COX-2 mediated disorder, the composition comprising a backing sheet that is flexibly conformable to the area of skin, the backing sheet having opposing surfaces that are respectively distal and proximal to the skin when applied; and a coating on the proximal surface of the backing sheet, said coating comprising (a) an adhesive, (b) an active agent comprising a selective COX-2 inhibitory sulfonamide drug of low water solubility, and (c) a solvent system for the active agent, wherein the active agent is in a therapeutically effective total amount and the solvent system is selected with regard to composition and amount thereof to be effective to maintain the active agent substantially completely in solubilized form.

2. The composition of claim 1 wherein the selective COX-2 inhibitory sulfonamide drug is a compound having the structural formula 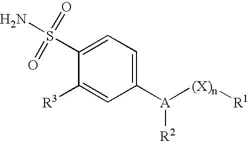
wherein:
A is a substituent selected from partially unsaturated or unsaturated heterocyclic and partially unsaturated or unsaturated carbocyclic rings;
X is O, S or CH₂;
n is 0 or 1;
R¹ is at least one substituent selected from heterocyclyl, cycloalkyl, cycloalkenyl and aryl groups, and is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio groups;
R² is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl groups, R² being optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio groups; and
R³ is selected from hydrido and halo radicals.

3. The composition of claim 2 wherein, in the formula for the selective COX-2 inhibitory sulfonamide drug, A is a pyrazole or isoxazole ring.

4. The composition of claim 1 wherein the selective COX-2 inhibitory sulfonamide drug is selected from the group consisting of celecoxib, deracoxib and valdecoxib.

5. The composition of claim 1 wherein the selective COX-2 inhibitory sulfonamide drug is valdecoxib.

6. The composition of claim 1 wherein the solvent system comprises N-methyl-2-pyrrolidone.

7. The composition of claim 1 wherein the coating further comprises one or more skin permeation enhancers.

8. The composition of claim 1 that further comprises a peelable release liner that, prior to application to the skin, is adjacent to the layer that contains the adhesive.

9. The composition of claim 1 wherein the coating comprises a layer having the active agent dispersed in a lipophilic matrix that comprises the adhesive and the solvent system.

10. The composition of claim 9 wherein the coating comprises about 0.1% to about 10% by weight of the active agent and about 0.5% to about 10% by weight ofthe solvent system.

11. The composition of claim 10 wherein the coating further comprises one or more crystallization inhibitors in a total amount up to about 30% by weight.

12. The composition of claim 10 wherein the coating further comprises one or more skin permeation enhancers in a total amount up to about 20% by weight.

13. The composition of claim 9 wherein the adhesive comprises about 10% to about 30% by weight of a styrene-isoprene-styrene block copolymer, about 20% to about 60% by weight of a tackifier resin, about 5% to about 20% by weight of a liquid rubber, about 10% to about 50% by weight of a softening agent and about 0.1% to about 5% by weight of an antioxidant.

14. The composition of claim 9 wherein the coating comprises
valdecoxib, 0.2-7% by weight;
N-methyl-2-pyrrolidone, 1-20% by weight;
crotamiton, 0-10% by weight;
polyvinylpyrrolidone, 0-20% by weight; and
oleic acid, 0-10% by weight;
in an adhesive matrix comprising a styrene-isoprene-styrene block copolymer, a tackifier resin, a liquid rubber, a softening agent and an antioxidant.

15. The composition of claim 14 wherein the coating comprises
valdecoxib, 0.5-5% by weight;
N-methyl-2-pyrrolidone, 2-10% by weight;
crotamiton, 0-5% by weight;
polyvinylpyrrolidone, 1-10% by weight; and
oleic acid, 0.5-5% by weight;
in said adhesive matrix.

16. The composition of claim 1 wherein the coating comprises a reservoir layer, adjacent to the backing sheet, wherein the active agent is dispersed in a hydrophilic matrix.

17. The composition of claim 16 wherein the reservoir layer further comprises the adhesive.

18. The composition of claim 16 wherein said reservoir layer is in a form of an aqueous gel.

19. The composition of claim 18 wherein the aqueous gel comprises about 0.1% to about 2% by weight of the active agent, about 0.5% to about 10% by weight of the solvent system and about 1% to about 20% by weight of the adhesive.

20. The composition of claim 19 wherein the aqueous gel further comprises one or more thickeners in a total amount up to about 10% by weight.

21. The composition of claim 19 wherein the aqueous gel further comprises one or more humectants in a total amount up to about 60% by weight.

22. The composition of claim 19 wherein the aqueous gel further comprises one or more skin permeation enhancers in a total amount up to about 20% by weight.

23. The composition of claim 19 wherein the aqueous gel further comprises one or more preservatives in a total amount up to about 1% by weight.

24. The composition of claim 18 wherein the aqueous gel comprises
valdecoxib, 0.2-1.5% by weight;
N-methyl-2-pyrrolidone, 1-15% by weight;
crotamiton, 0.2-10% by weight;
oleic acid, 0-10% by weight;
polyacrylate adhesive, 1-10% by weight as solids;
organic acid, 0-5% by weight;
glycerol, 5-50% by weight;
sodium polyacrylate, 0-15% by weight;
carmellose sodium, 0-15% by weight;
hydroxypropylcellulose, 0-10% by weight;
polyvalent salt, 0-2% by weight;
disodium edetate, 0-1% by weight;
propylene glycol, 0-30% by weight;
paraben, 0-1% by weight;
castor oil, 0-5% by weight;
surfactant, 0-5% by weight;
urea, 0-10% by weight;
menthol, 0-5% by weight; and
water and other optional ingredients, balance to 100% by weight.

25. The composition of claim 18 wherein the aqueous gel comprises valdecoxib, 0.3-1% by weight;
N-methyl-2-pyrrolidone, 2-10% by weight;
crotamiton, 0.5-5% by weight;
oleic acid, 0.5-5% by weight;
polyacrylate adhesive, 1.5-7% by weight as solids;
organic acid, 0-2% by weight;
glycerol, 10-40% by weight;
sodium polyacrylate, 0-8% by weight;
carmellose sodium, 0-8% by weight;
hydroxypropylcellulose, 0-6% by weight;
polyvalent salt, 0-1% by weight;
disodium edetate, 0-0.5% by weight;
propylene glycol, 0-20% by weight;
paraben, 0.05-0.5% by weight;
castor oil, 0-2% by weight;
surfactant, 0-2% by weight;
urea, 0-5% by weight;
menthol, 0-2% by weight; and
water and other optional ingredients, balance to 100% by weight.

26. A method of local treatment of a site of pain and/or inflammation in a subject, the method comprising a step of applying to a skin surface of the subject a pharmaceutical composition that comprises a backing sheet that is flexibly conformable to the skin surface, said backing sheet having opposing surfaces that are respectively distal and proximal to the skin when applied, and a coating on the proximal surface of the backing sheet, said coating comprising (a) an adhesive, (b) an active agent comprising a selective COX-2 inhibitory sulfonamide drug of low water solubility, and (c) a solvent system for the active agent, wherein the active agent is in a therapeutically effective total amount and the solvent system is selected with regard to composition and amount thereof to be effective to maintain the active agent substantially completely in solubilized form; and a step of leaving the composition in place for a time period effective to permit delivery of a locally therapeutic amount of the active agent.

27. A method of systemic treatment of a subject having a COX-2 mediated disorder, the method comprising a step of applying to a skin surface of the subject a pharmaceutical composition that comprises a backing sheet that is flexibly conformable to the skin surface, said backing sheet having opposing surfaces that are respectively distal and proximal to the skin when applied, and a coating on the proximal surface of the backing sheet, said coating comprising (a) an adhesive, (b) an active agent comprising a selective COX-2 inhibitory sulfonamide drug of low water solubility, and (c) a solvent system for the active agent, wherein the active agent is in a therapeutically effective total amount and the solvent system is selected with regard to composition and amount thereof to be effective to maintain the active agent substantially completely in solubilized form; and a step of leaving the composition in place for a time period effective to permit transdermal delivery of a therapeutic amount of the active agent.
